(19) United States
(12) Patent Application Publication
(10) Pub. No.: US 2026/0158254 A1
(43) Pub. Date: Jun. 11, 2026

(54) MICRONEEDLE PATCH AND MANUFACTURING METHOD THEREOF

(71) Applicant: DARWIN PRECISIONS CORPORATION, Hsinchu County (TW)

(72) Inventor: Yi-Chen Liu, Hsinchu County (TW)

(21) Appl. No.: 19/178,877

(22) Filed: Apr. 15, 2025

(30) Foreign Application Priority Data
Oct. 4, 2024 (TW) 113137919

(51) Int. Cl. A61M 37/00; A61K 9/00; A61K 9/70

(52) U.S. Cl. CPC A61M 37/0015; A61K 9/0021; A61K 9/703; A61M 2037/0007; A61M 2037/0023; A61M 2037/0046; A61M 2037/0053

(57) ABSTRACT

A microneedle patch and a manufacturing method thereof are provided. The microneedle patch includes a microneedle layer. The microneedle layer has a total area, and the microneedle layer has a first region. The first region has a first area less than or equal to the total area. A material of the microneedle layer includes a biocompatible material, a thermochromic material, a photochromic material, a thermochromic material-induced reactant, a photochromic material-induced reactant, or a combination thereof.

### FIELD OF THE INVENTION

The present invention relates to a transdermal delivery field, in particular to a microneedle patch and a manufacturing method thereof.

### BACKGROUND OF THE INVENTION

Microneedle patches are commonly seen in medical field, medical aesthetic field and others. In order to meet the requirements of the microneedle patch in terms of appearance and variability, there is a manufacturing method of the microneedle patch, which uses a laser to drill microholes in a mold according to the pattern design; however, each time a new pattern is designed, a mold has to be remanufactured, which is costly and not conducive to mass production. Another manufacturing method uses 3D printing technology to manufacture a plug and a common mold to form microneedles arranged according to a design pattern. However, because there is a new pattern design, a new plug needs to be made, and cost of the plug manufacturing is high, and degree of fitness between the plug and the mold also needs to be improved, so this method is not conducive to commercial production.

### SUMMARY OF THE INVENTION

The present invention provides a microneedle patch, which has variability in appearance and can be used by users to change the shape to achieve various visual effects. It can also provide marking, tracking, indicating and other functions for identity recognition and medical assistance.

The present invention also provides a method for manufacturing a microneedle patch, which can reduce manufacturing costs, help improve the yield of microneedles, and facilitate mass production of the microneedle patches.

In order to achieve one, part, or all of the above purposes, or other purposes, an embodiment of the present invention provides the microneedle patch, which includes a microneedle layer with a total area. The microneedle layer includes a first region. The first region has a first area less than or equal to the total area. A material of the microneedle layer includes a biocompatible material, a thermochromic material, a photochromic material, a thermochromic material-induced reactant, a photochromic material-induced reactant, or a combination thereof.

In one embodiment of the present invention, the material of the microneedle layer further includes a tattoo pigment.

In one embodiment of the present invention, an amount of the thermochromic material, the photochromic material, the thermochromic material-induced reactant, the photochromic material-induced reactant, or the combination thereof accounts for 0.1 to 40 wt % of the material of the microneedle layer.

In one embodiment of the present invention, the amount of the thermochromic material, the photochromic material, the thermochromic material-induced reactant, the photochromic material-induced reactant, or the combination thereof accounts for 2 to 35 wt % of the material of the microneedle layer.

In one embodiment of the present invention, the amount of the thermochromic material, the photochromic material, the thermochromic material-induced reactant, the photochromic material-induced reactant, or the combination thereof accounts for 10 to 25 wt % of the material of the microneedle layer.

In one embodiment of the present invention, the material of the microneedle layer further includes the tattoo pigment, and an amount of the thermochromic material, the photochromic material, the thermochromic material-induced reactant, the photochromic material-induced reactant, the tattoo pigment, or the combination thereof accounts for 0.1 to 40 wt % of the material of the microneedle layer.

In order to achieve one, part, or all of the above purposes, or other purposes, an embodiment of the present invention also provides a method for manufacturing a microneedle patch, including steps of: providing a mold, wherein an internal space of the mold includes a bottom surface, a plurality of tapered holes is formed on the bottom surface, and the tapered holes are spaced apart from each other and extend in a direction away from the bottom surface; providing the biocompatible material, wherein the biocompatible material mixes with the thermochromic material, the photochromic material, or a combination thereof to form a first mixture; dispensing the first mixture to the mold and forming the microneedle layer, wherein the microneedle layer includes a plurality of microneedles spaced apart from each other; arranging a mask above the microneedle layer, wherein the mask has a pattern region, and the pattern region is projected on the microneedle layer; and providing a heat source, a cold source, a light source, or a combination thereof, and applying a temperature change, light, or a combination thereof to the microneedle layer across the mask so that the microneedle layer displays a pattern, wherein the pattern corresponds to the pattern region of the mask.

In order to achieve one, part, or all of the above purposes, or other purposes, an embodiment of the present invention also provides a microneedle patch, which is prepared by a method including the above steps.

The present invention can be used in combination with the mask, the heat source, the cold source, or the light source because the present invention adopts the thermochromic material, the photochromic material, the thermochromic material-induced reactant, the photochromic material-induced reactant, or the combination thereof, which is conducive to simplifying the manufacturing process.

Other objectives, features and advantages of the invention will be further understood from the further technological features disclosed by the embodiments of the invention wherein there are shown and described preferred embodiments of this invention, simply by way of illustration of modes best suited to carry out the invention.

### BRIEF DESCRIPTION OF THE DRAWINGS

The present invention will become more readily apparent to those ordinarily skilled in the art after reviewing the following detailed description and accompanying drawings, in which:

FIG. 1 is a schematic flow chart of a method for manufacturing a microneedle patch according to an embodiment of the present invention;

FIGS. 2, 3A, 3B, and 4-8 are schematic diagrams showing the steps of the method for manufacturing the microneedle patch according to an embodiment of the present invention;

FIG. 9 is a schematic top view of the microneedle patch according to the first embodiment of the present invention;

FIG. 10 is a three-dimensional schematic diagram of the microneedle patch according to a second embodiment of the present invention;

FIG. 11 is a three-dimensional schematic diagram of a microneedle patch according to a third embodiment of the present invention; and

FIG. 12 is a schematic top view of a microneedle patch according to the third embodiment of the present invention.

### DETAILED DESCRIPTION OF PREFERRED EMBODIMENTS

The aforementioned and other technical contents, features and effects of the present invention will be clearly presented in the following detailed description of a preferred embodiment with reference to the drawings. The direction terms mentioned in the following embodiments only refer to the directions of the attached drawings. Accordingly, the directional terms used are illustrative and not limiting of the invention. In addition, terms such as “first” and “second” mentioned in this specification or the scope of the patent application are only used to name elements or to distinguish different embodiments or scopes, and are not used to limit the upper or lower limit on the number of elements.

The present invention provides a microneedle patch and a manufacturing method thereof. The microneedle patch is preferably identifiable, for example, can display a pattern or text. Alternatively, the microneedle patch itself has a specific shape, and the shape of the microneedle patch can reflect a specific pattern or text. The microneedle patch of the present invention can also be used as a tattoo patch.

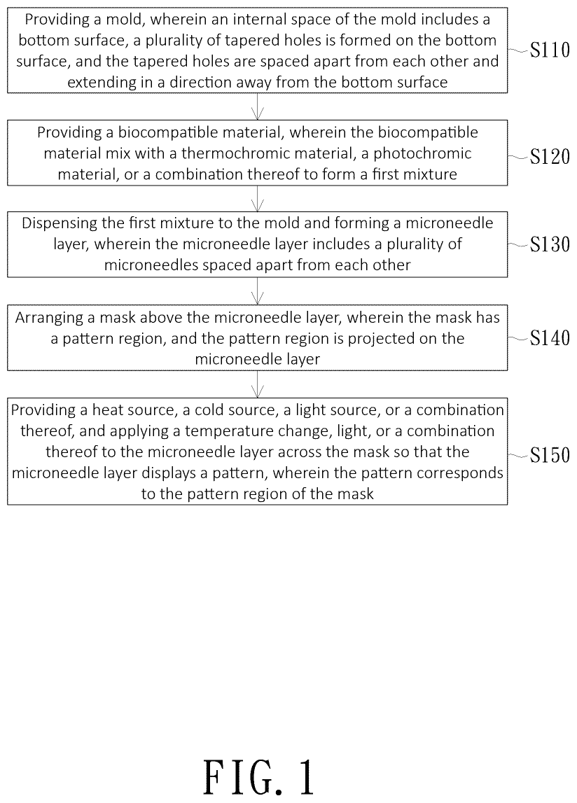

FIG. 1 is a schematic flow chart of the method for manufacturing the microneedle patch according to an embodiment of the present invention. As shown in FIG. 1, the manufacturing method can include a step S110: providing a mold, wherein an internal space of the mold includes a bottom surface, a plurality of tapered holes is formed on the bottom surface, and the tapered holes are spaced apart from each other and extend in a direction away from the bottom surface; a step S120: providing a biocompatible material, wherein the biocompatible material mixes with a thermochromic material, a photochromic material, or a combination thereof to form a first mixture; a step S130: dispensing the first mixture to the mold and forming a microneedle layer, wherein the microneedle layer includes a plurality of microneedles spaced apart from each other; a step S140: arranging a mask above the microneedle layer, wherein the mask has a pattern region, and the pattern region is projected on the microneedle layer; a step S150: providing a heat source, a cold source, a light source, or a combination thereof, and applying a temperature change, light, or a combination thereof to the microneedle layer across the mask so that the microneedle layer displays the pattern, wherein the pattern corresponds to the pattern region of the mask. Each step is further explained hereinafter.

Step S110:

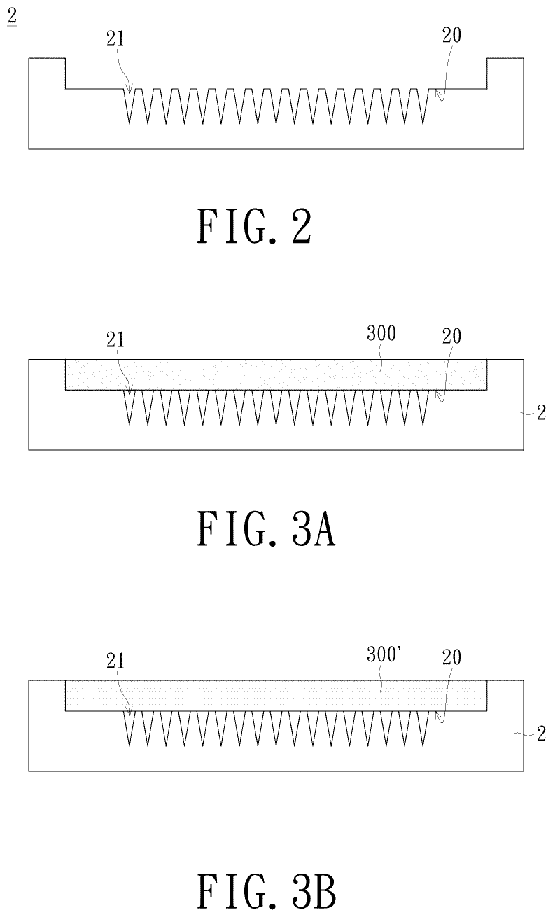

The mold in the step S110 can be, for example, a master mold, and is preferably formed of a polymeric material. In some embodiments, the master mold can be formed by, for example, using a male mold and polymeric materials such as polyethylene, polypropylene, polylactic acid, polybutylene succinate, and polydimethylsiloxane. The male mold (not shown) can have multiple cone-shaped or needle-shaped structures, which reflect the shape of the microneedles of the microneedle patch. The cone-shaped or the needle-shaped structures of the male mold can be, for example, a cone, a triangular pyramid, or a quadrangular pyramid, but are not limited thereto. The mold as the master mold is formed with holes complementary to the cone-shaped or the needle-shaped structures through the male mold. As shown in FIG. 2, the internal space of the mold 2 includes a bottom surface 20, and a plurality of tapered holes 21 is formed on the bottom surface 20, and the tapered holes are spaced apart from each other and extend in the direction away from the bottom surface 20. A shape of the tapered holes 21 corresponds to the shape of the microneedles, and a depth of the tapered holes 21 can reflect a height of the microneedles. In a preferred embodiment of the present invention, the depth of the tapered holes 21 can be 100-2000 μm, and the height of the microneedles can be, for example, 100-2000 μm.

Step S120:

The microneedle patch of the present invention is, in principle, a patch in which the microneedles are soluble and/or biodegradable in living organisms, and a main component constituting the microneedles is the biocompatible material. The biocompatible material can include hyaluronic acid, polyvinylpyrrolidone, polyvinyl alcohol, hydroxyethylcellulose, carboxymethylcellulose, hydroxypropylmethylcellulose, pullulan gum, collagen, xanthan gum, pectin, polyglutamic acid, short-chain polypeptides, sodium alginate, maltodextrin, cyclodextrin, propylene glycol alginate, gelatin, gum arabic, methylcellulose, polysaccharides such as dextran, polymannose, short-chain polysaccharides, disaccharides such as trehalose, sucrose, lactose, monosaccharides such as glucose, polyols such as mannitol, sorbitol, or a combination thereof, but is not limited thereto. For example, other high molecular polymer materials and low molecular weight materials other than those listed can also be used. In addition, the materials constituting microneedles preferably include high molecular polymer materials and low molecular weight materials, wherein low molecular weight materials can be used to adjust properties. During a preparation stage, the biocompatible material can preferably be in a form of solution or gel. After being injected into the mold 2, the biocompatible material can be heated or cooled to form a shape. In a preferred embodiment of the present invention, the biocompatible material includes, for example, hyaluronic acid.

The biocompatible material is homogeneously mixed with the thermochromic material, the photochromic material, or the combination thereof. That is, one of the thermochromic material and the photochromic material can be selected and mixed with the biocompatible material, or both can be used together to form a first mixture 300. The thermochromic material is a material that can change color under a specific temperature change. The specific temperature change can be, for example, how many degrees the temperature rises, how many degrees the temperature drops, or reaches a specific temperature range, but is not limited thereto. In addition, a color change can be reversible or irreversible. The thermochromic material includes any known or yet-to-be-developed materials that can change color or cause color to change. For example, they can include color-changing liquid crystals, spironolactone, fluoroline, spiropyran, fulgide, triarylmethane, bis(methyldimethylamino)tetrachloronickelate (II), bis(ethyldiethylamino)tetrachlorocuprate (II), tetraorganodiarsenic, tetraorganoditin, tetraorganodibismuth, ammonium metavanadate, manganese violet, metal oxides, metal sulfides, iodides, or a combination thereof, but are not limited thereto. The metal oxides include, for example, titanium dioxide, zinc oxide, indium oxide, lead oxide, chromium oxide, aluminum oxide, and vanadium dioxide. The metal sulfides include, for example, zinc sulfide. And, the iodides include, for example, mercury iodide, silver mercury iodide, and copper iodide, but are not limited thereto. The thermochromic material can be mixed into the biocompatible material in the form of, for example, powder, liquid, microcapsules, or a combination thereof. Several embodiments of the present invention use thermochromic microcapsules with gold particles (insilico), such as Chameleon T series, Bichrom T series, Irreversibility T series, and SpyBall. The thermochromic material allows the microneedles or the microneedle layer (described below) composed of the first mixture 300 to undergo color development (from originally transparent and colorless to color), decolorization (from original color to transparent and colorless), and color conversion (from original color to another color) under temperature changes. The color changes are not limited to being visible to the naked eyes.

The photochromic material is a material that can change color or cause color to change under specific light. The color change is not limited to being visible to the naked eyes. The specific light can be, for example, UV light, infrared light, or visible light, but is not limited thereto. The color change can likewise be reversible or irreversible. The photochromic material includes any known or yet-to-be-developed material that can undergo color change, including, for example, triarylmethane, spiropyran, spirooxazine, diarylethene, azobenzene, nitrone, fulgide, naphthopyrans, quinones, chlorides, halides, hydroxides, metal complexes, coordination compounds, dark color-changing minerals, or a combination thereof, but are not limited thereto. The azobenzene is, for example, azovinylbenzene. The chloride is, for example, silver chloride. The halide is, for example, silver halide or zinc halide. The hydroxide is, for example, yttrium hydroxide. And, the coordination compound can be sodium nitroprusside or ruthenium sulfoxide. The photochromic material can be mixed with the biocompatible material, e.g. in the form of powders, liquids, microcapsules, or a combination thereof. For example, photochromic microcapsules are used in several embodiments of the present invention. The photochromic material enables the microneedles or the microneedle layer (described below) composed of the first mixture 300 to develop, decolorize, and convert color under specific light. An amount of the thermochromic material, the photochromic material, or the combination thereof can account for 0.1 to 40 wt % of the first mixture 300, such as 0.1 wt %, 1 wt %, 5 wt %, 10 wt %, 15 wt %, 20 wt %, 25 wt %, 30 wt %, 35 wt %, and 40 wt %, wherein when the thermochromic material and the photochromic material are used in combination, a sum of two amounts also accounts for 0.1 to 40 wt % of the first mixture 300. In the embodiment of the present invention, for example, the sum of two amounts of the thermochromic material and the photochromic material accounts for 18 wt % of the first mixture 300, and the thermochromic material and the photochromic material can be combined in a ratio of, for example, 5:1. That is, the amounts of the thermochromic material and the photochromic material respectively account for 15 wt % and 3 wt % of the first mixture 300, but are not limited thereto.

In principle, the amount of the thermochromic material, the photochromic material, or the combination thereof shall not be too high; one of the reasons is that the amount of the biocompatible material will be relatively low, which will lead to insufficient strength, loss of microneedles, or other instability of physical properties, affecting the efficacy of microneedles to puncture the skin. On the other hand, the amount of the thermochromic material, the photochromic material, or the combination thereof shall not be too low, as this may make the color too light and not conducive to identification. In the preferred embodiment of the present invention, the thermochromic material, the photochromic material, or the combination thereof further is 2 to 35 wt %, such as 2 wt %, 4 wt %, 7 wt %, 11 wt %, 15 wt %, 19 wt %, 23 wt %, 27 wt %, 31 wt %, and 35 wt %. For example, hyaluronic acid and photochromic powder with a mixing ratio of 95:5 can be used as the material of the microneedle layer. In the better embodiment of the present invention, the amount of the thermochromic material, the photochromic material, or the combination thereof is 10 to 25 wt %, such as 10 wt %, 12 wt %, 14 wt %, 16 wt %, 18 wt %, 20 wt %, 22 wt %, 24 wt %, and 25 wt %. Depending on the type and content of the thermochromic material and the photochromic material, the step S120 can further include evenly dispersing the thermochromic material, the photochromic material, or the combination thereof in the biocompatible material. Further, when the amount of the thermochromic material, the photochromic material, or the combination thereof is, for example, greater than 10 wt %, the formulation of the biocompatible material can be adjusted to facilitate the even dispersion of the thermochromic material, the photochromic material, or the combination thereof in the microneedles, thereby achieving better color-changing results.

In addition to the thermochromic material and the photochromic material, an embodiment of the present invention can further add other ingredients to the biocompatible material. For example, the other ingredients can include the above-mentioned low molecular weight materials, catalysts, substrates for the thermochromic material or the photochromic material, donors for the thermochromic material or the photochromic material such as proton donors, and additives for adjusting the properties of microneedles, but are not limited thereto.

In a preferred embodiment of the present invention, the thermochromic material, the photochromic material, the combination thereof, or derivatives thereof (described below) preferably also has tattoo effects. At this time, the thermochromic material, the photochromic material, the combination thereof, or the derivatives thereof can be injected into the skin through microneedles to achieve the tattoo effects and further achieve the purpose of decoration or marking. The step S120 can further include mixing a tattoo pigment into the first mixture 300 to form a second mixture 300′. The tattoo pigment includes any known or a yet-to-be-developed material that can display a specific color pattern on the skin. For example, the tattoo pigment can include mercury sulfide (cinnabar), cadmium sulfoselenide (cadmium red), iron oxide, carmine, cochinilla, chromium oxide, hydrous chromium oxide, copper chloride, chromium trioxide, emerald green, cobalt aluminate, indigo, phthalocyanine blue, cadmium sulfide, lemon yellow, chromium zinc, hydrate of ferric oxide, logwood, carbon, iron monoxide, ferric tetroxide, black ink, cadmium salt, venetian red, zinc oxide, barium sulfate, titanium dioxide, lead carbonate, ammonium manganese(3+) diphosphate, various aluminum salts of quinacridone, dihydroxyquinoloacridine, dioxazine, triazole, naphthalenedione, or a combination thereof. The tattoos are not limited to permanent or temporary tattoos.

The tattoo pigment, the thermochromic material, the photochromic material, or a combination thereof can account for 0.1 to 40 wt % of the second mixture 300′, such as 0.1 wt %, 1 wt %, 5 wt %, 10 wt %, 15 wt %, 20 wt %, 25 wt %, 30 wt %, 35 wt %, and 40 wt %. In the embodiment of the present invention, for example, a total amount of the tattoo pigment, the thermochromic material, and the photochromic material accounts for 18 wt % of the second mixture 300′, wherein the tattoo pigment, the thermochromic material, and the photochromic material can be combined in a ratio of, for example, 1:3:2; that is, the amounts of the tattoo pigment, the thermochromic material and the photochromic material respectively account for 3 wt %, 9 wt %, and 6 wt % of the second mixture 300′, but are not limited thereto.

Step S130:

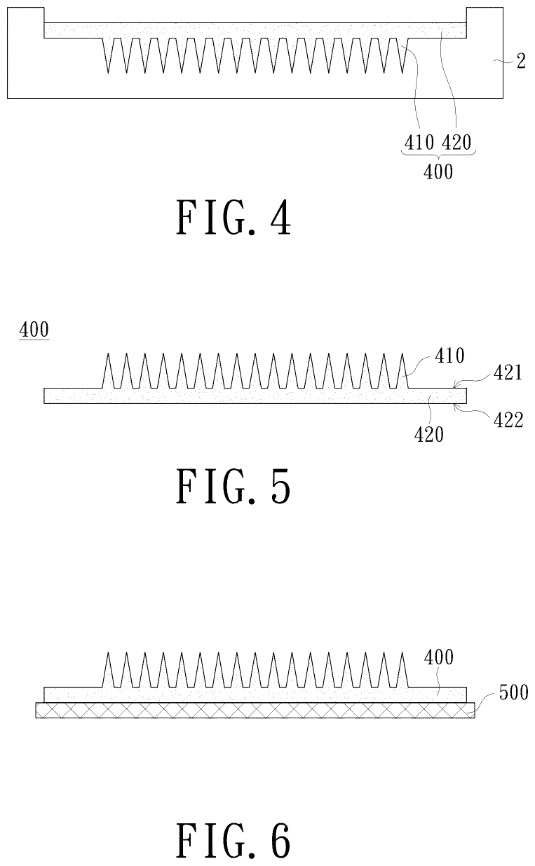

The step S130 uses the mold 2 of the step S110 and the first mixture 300 or the second mixture 300′ of the step S120. As shown in FIG. 3A, during dispensing, the first mixture 300 is first injected into the internal space of the mold 2, and preferably, the liquid level of the first mixture 300 can be horizontal and parallel to the bottom surface 20. Or as shown in FIG. 3B, the second mixture 300′ is injected into the internal space of the mold 2. As shown in FIG. 4, the first mixture 300 (or the second mixture 300′; the following is straightforward explained with the first mixture 300), for example, can enter the tapered holes 21 from the bottom surface 20 due to an influence of gravity. The tapered holes 21 are filled with the first mixture 300, and preferably are completely filled. As the first mixture 300 enters the tapered holes 21, a liquid level of the first mixture 300 above the bottom surface 20 can decrease.

Whether the first mixture 300 completely fills the tapered holes 21 can be checked by an instrument or observed by whether there is a liquid level change. Afterwards, the first mixture 300 can be solidified and formed in the mold 2 to form a plurality of microneedles 410 and a base layer 420. The microneedles 410 and the base layer 420 form the microneedle layer 400, wherein the plurality of microneedles 410 is spaced apart from each other and arranged on one side of the base layer 420. Generally speaking, the materials constituting the microneedles 410 and the base layer 420 are the same, such as a mixture of hyaluronic acid and photochromic powder, but are not limited thereto. In some embodiments of the present invention, the materials of the microneedles 410 and the base layer 420 can also be different; for example, the tapered holes 21 can be filled with the first mixture 300 containing the thermochromic material, the photochromic material, or the combination thereof together with the biocompatible material, and then the bottom surface 20 can be covered with the biocompatible material to form the plurality of microneedles 410 containing the thermochromic material, the photochromic material, or the combination thereof, and the base layer 420 without containing the thermochromic material, the photochromic material, or the combination thereof. Alternatively, the tapered holes 21 can be filled with the second mixture 300′ containing the tattoo pigment, and then the bottom surface 20 can be covered with the biocompatible material to form the plurality of microneedles 410 containing the tattoo pigment, and the base layer 420 without containing the tattoo pigment.

An embodiment of the manufacturing method of the present invention can further include a step S135: separating the microneedle layer 400 from the mold 2. The separated microneedle layer 400 is shown in FIG. 5. In the embodiment of the present invention, the microneedles 410 can be cones, triangular pyramids, or quadrangular pyramids, but are not limited thereto, and the height can be, for example, 100 to 2000 μm. The side of the microneedles 410 on the base layer 420 can be a first side 421, and the side opposite to the first side 421 is a second side 422. As shown in FIG. 6, a carrier layer 500 can be further arranged on the second side 422. The carrier layer 500 can be made of a tougher material, such as film or cloth, which can carry the microneedle layer 400 and is convenient for the user to hold the microneedle patch.

Step S140:

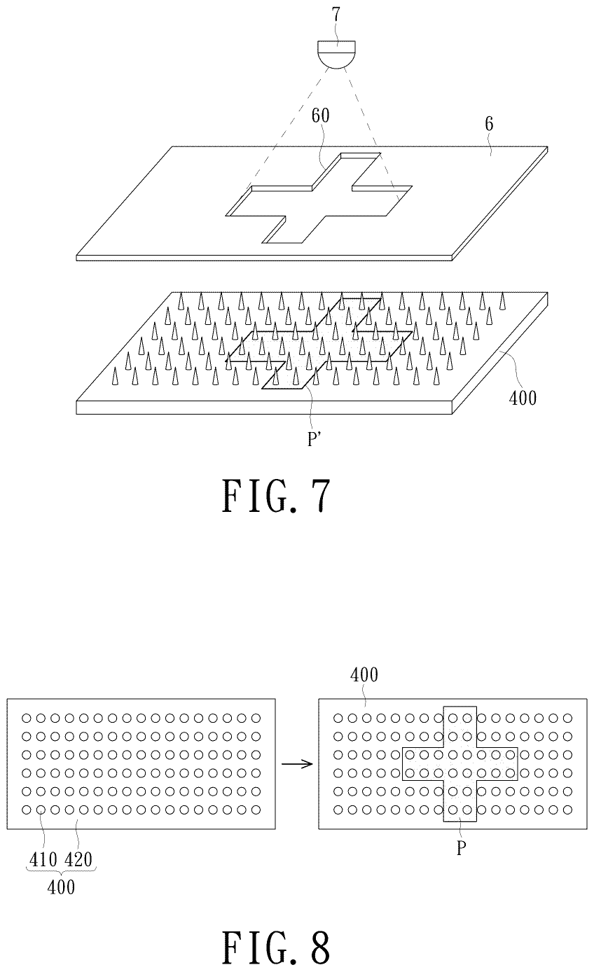

As shown in FIG. 7, a mask 6 used in the step S140 has a pattern region 60. In the embodiment of the present invention, the pattern region 60 can be a hollow region on the mask 6, but is not limited thereto. For example, the solid part of the mask 6 outside the hollow region can also present a pattern and be another pattern region. In the preferred embodiment of the present invention, the hollow region on the mask 6 is the pattern region 60. The hollow region is not limited to the cross shape as shown in FIG. 7. For example, the hollow region can also be geometric figures, irregular shapes, or text, and the present invention is not limited thereto.

The mask 6 in the embodiment of the present invention preferably has properties of heat insulation, light blocking, or a combination thereof. Therefore, when there is the heat source, the cold source, or the light source on one side of the mask 6, the mask 6 can partially block heat, cold, or light. That is, high, low temperatures, or light are preferably transmitted from the hollow region to the other side of the mask 6, and conversely, the other side corresponding to the part outside the hollow region is preferably not affected by the temperatures and the light. A material of the mask 6 can be, for example, metal, organic silicon, inorganic silicon, paper, plastic, or resin, but is not limited thereto.

In the embodiment of the present invention, as shown in FIG. 7, the mask 6 can be arranged on a side of the microneedle layer 400 with the microneedles 410, and the pattern region 60 is projected on the microneedles 410 and the base layer 420. The mask 6 may have no directionality; for example, when the pattern region 60 thereon is a completely symmetrical pattern, it basically makes no difference which side of the mask 6 faces the microneedle layer 400. The mask 6 can also have directionality; for example, when the pattern region 60 thereon is an asymmetrical pattern such as the letter “B”, then the mask 6 will produce different projections P′ with different faces facing the microneedle layer 400. Therefore, when the mask 6 is arranged on the side of the microneedle layer 400 with the microneedles 410, the step S140 can further include making a direction of the projection P′ opposite to the tattoo pattern that the microneedle patch 40 intends to be achieved, such as oppositely exchanging left and right. On the contrary, when the mask 6 is arranged on the base layer 420 side of the microneedle layer 400, the projection P′ can be in a same direction as the tattoo pattern that the microneedle patch 40 intends to be achieved. A number of the pattern region 60 is not limited to one; that is, the mask 6 can have multiple pattern regions 60. In addition, the pattern region 60 can be a single continuous region or multiple discontinuous regions.

Step S150:

The step S150 can select a cold/a heat source or a light source 7 according to the preceding steps, and the cold source or the heat source can be further determined according to the specific content of the thermochromic material, or a wavelength of the light source can be further determined according to the specific content of the photochromic material. For example, the thermochromic material changes color when the temperature drops, the cold source is usually selected. The cold source or the heat source can cool or heat the microneedle layer 400 through, for example, air conduction or electromagnetic waves that across the mask 6. Specifically, the cold/the heat source includes but are not limited to a cold/a hot air, liquid nitrogen, microwaves, and lasers. Since the mask 6 body is preferably thermally insulated, the cold/the heat source passes through the pattern region 60 and causes the projection range P′ on the microneedle layer 400 to be cooled or heated. As a result, as shown in FIG. 8, the color of the microneedle layer 400 that ranges within the projection P′ is caused to change and a pattern P is revealed. In some embodiments of the present invention, the mask 6 is a stamping processing polydimethylsiloxane (PDMS) mask, and the heat source 7 can be, for example, a 60-350° C. heat gun.

On the other hand, the light source 7 can be a light source of various wavelengths, including but not limited to ultraviolet light, infrared light, and visible light, which illuminates the microneedle layer 400 across the mask 6. Since the mask 6 itself is preferably light-blocking, the light passes through the pattern region 60 and illuminates the range within the projection P′ on the microneedle layer 400, causing the microneedle layer 400 to change color and reveal the pattern P. In some embodiments of the present invention, the mask 6 is a laser-engraved metal mask, and the light source 7 can be, for example, a 5 W 200-380 nm ultraviolet lamp. Depending on the type of the photochromic material, the wavelength range of the light emitted by the light source 7 preferably includes ultraviolet light of 365 nm.

The above-mentioned “color change of the thermochromic material/the photochromic material” can be caused by changes in the thermochromic material or the photochromic material themselves, such as structural changes, under an influence of the temperature change or light. In the present invention, the modified thermochromic material and the modified photochromic material are called derivatives. The derivatives can be formed from the thermochromic material, the photochromic material, and the other ingredients. For example, the photochromic material can form bonds with the other ingredients to produce the derivatives after being illuminated, but is not limited thereto. “The thermochromic material/the photochromic material causes color change” can be caused by products generated by the thermochromic material, the photochromic material, and the other ingredients under the action of temperature change or light (hereinafter referred to as a product after temperature change or a product after light exposure). For example, the photochromic material can catalyze a substrate thereof to produce a product when exposed to light. The photochromic material itself only acts as a catalyst to promote color changes in the other ingredients, but is not limited thereto. In the present invention, the derivatives of the thermochromic material and the products after temperature change can be called a thermochromic material-induced reactant, and the derivatives of photochromic material and the products after light exposure can be called a photochromic material-induced reactant. However, in some embodiments of the present invention, the thermochromic material-induced reactant also means the entirety of the thermochromic material, the derivatives thereof, or the products after temperature change therefor, and is not limited thereto. For example, the thermochromic material-induced reactant can be the thermochromic material such as spironolactone, spiropyran, fulgide and proton donor thereof such as the entirety of bisphenol A, parabens, 1,2,3-triazole derivatives, and 4-hydroxycoumarin. In addition, the photochromic material-induced reactant also means the entirety of the photochromic material, the derivatives thereof, or the products after light exposure therefor, and is not limited thereto. An effective time of color change of the thermochromic material-induced reactant and the photochromic material-induced reactant can be temporary or permanent. For example, a color can be developed temporarily and maintained for a period of time. After this period of time the color can be lost gradually or immediately.

In the embodiment of the present invention, the thermochromic material and the photochromic material that can generate the products after temperature change or the products after light exposure can further have the tattoo effects, and the products thereof can also have the tattoo effects. In addition, the thermochromic material and the photochromic material, which can form the above-mentioned derivatives under the action of temperature change or light, can further have the tattoo effects, and the derivatives thereof can also have the tattoo effects. In this case, as shown in FIG. 8, the microneedle layer 400 has an effect equivalent to the composition of tattoo pigment within the range of the pattern P thereof, and the pattern P better reflects the tattoo effect of the microneedle patch 40, which is substantially the same as the pattern P. The tattoo effect ingredients in the pattern P can be distributed only in the microneedles 410, or can be distributed only in the base layer 420, or can be distributed in both. The color produced by the tattoo effect ingredients on the skin is not limited to the same color as the pattern P.

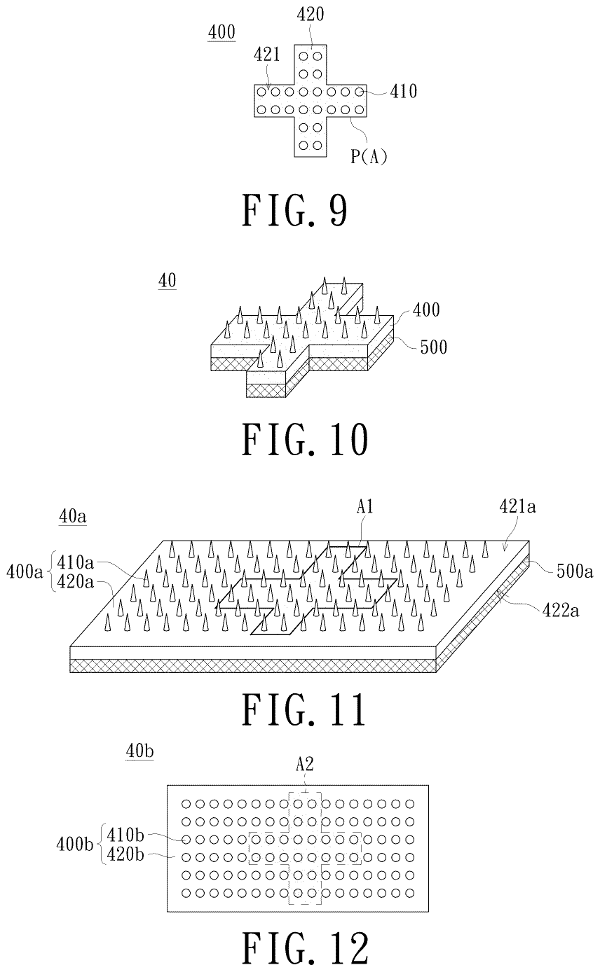

An embodiment of the manufacturing method of the present invention can further include a step S155: cutting the microneedle layer 400 according to the pattern P. The cut microneedle layer 400 can have a shape corresponding to the pattern P. As shown in the embodiment shown in FIG. 9, the pattern P is in a shape of a cross, and the microneedle layer 400 is also in the shape of a cross. Further, as shown in FIG. 10, the carrier layer 500 arranged on the second side 422 of the microneedle layer 400 can also have the same shape, but is not limited thereto. After completing the step S150 or the step S155, the microneedle patch 40 can be obtained. The microneedle patch 40 can be used as a tattoo patch because the microneedle patch 40 contains ingredients that have tattoo effects.

The present invention also provides a type of microneedle patch. As shown in an embodiment shown in FIG. 11, a microneedle patch 40a includes a microneedle layer 400a. The microneedle layer 400a also includes a base layer 420a and a plurality of microneedles 410a. The base layer 420a has a first side 421a and a second side 422a. The plurality of microneedles 410a is spaced apart on one side, such as the first side 421a. The microneedle patch 40a can further include a carrier layer 500a, and the microneedle layer 400a can be arranged on the carrier layer 500a on other side of the base layer 420a, such as the second side 422a.

The microneedle layer 400a can have a total area. The total area is related to a size and a shape of the microneedle layer 400a. The total area can also be equivalent to an area of the first side 421a or the second side 422a. In the embodiment of FIG. 11, the microneedle layer 400a is rectangular, so the total area can be approximately the product of the length and width of the rectangle. The microneedle layer 400a includes a first region A1. The first region A1 is preferably located on a side where the microneedles 410a are located, such as the first side 421a, or the first region A1 can also be located on the second side 422a. A number of the first region A1 is not limited to one; for example, there are multiple first regions A1 on the first side 421a. In addition, the first region A1 can be a single continuous region or can be multiple discontinuous regions. The first region A1 has a first area. The first area can be less than or equal to the total area. In the embodiment of FIG. 11, the first region A1 of the microneedle layer 400a generally has a cross-shaped pattern and is located within the range of the first side 421a. At this time, the first area is generally a cross-shaped area, and the first area is smaller than the total area. However, in some embodiments, the first area can be equal to the total area. Taking FIG. 9 as an example, the first side 421 is in a shape of a cross, the first region A is also in a shape of the cross, and the total area has the area of the cross, and an area of the first area also has the area of the cross and is equal to the total area.

A material of the microneedle layer 400a includes the biocompatible material along with the thermochromic material, the photochromic material, the thermochromic material-induced reactant, the photochromic material-induced reactant, or the combination thereof. That is, the microneedle layer 400a is made of the biocompatible material, and can contain any one, two, three, or all of the thermochromic material, the photochromic material, the thermochromic material-induced reactant, and the photochromic material-induced reactant. The definitions of the thermochromic material, the photochromic material, the thermochromic material-induced reactant and the photochromic material-induced reactant can be as described above and will not be repeated here. A method of constructing the microneedle layer 400a using the biocompatible material, the thermochromic material, the photochromic material, the thermochromic material-induced reactant, the photochromic material-induced reactant, or the combination thereof can be as described above; in addition, the microneedle layer 400a can be manufactured by the same method as described above. It is known that the thermochromic material, the photochromic material, the thermochromic material-induced reactant, the photochromic material-induced reactant, or the combination thereof can be distributed in the microneedles 410a and the base layer 420a of the microneedle layer 400a, or can be distributed only in the microneedles 410a or only in the base layer 420a.

In the embodiment of the present invention, the thermochromic material, the photochromic material, the thermochromic material-induced reactant, the photochromic material-induced reactant, or the combination thereof accounts for 0.1 to 40 wt % of the microneedle layer material, preferably 2 to 35 wt %, more preferably 10 to 25 wt %. In a preferred embodiment of the present invention, the thermochromic material is a thermochromic microcapsule, and the photochromic material is a photochromic microcapsule. In some embodiments of the present invention, the thermochromic material, the photochromic material, the thermochromic material-induced reactant, the photochromic material-induced reactant, or the combination thereof can have the tattoo effects.

In a preferred embodiment of the present invention, the thermochromic material-induced reactant, the photochromic material-induced reactant, or the combination thereof are further distributed in the plurality of microneedles 410a located in the first region A1, and can have the tattoo effects. Taking FIG. 11 as an example, the microneedles 410a ranging within the cross-shaped pattern contain the thermochromic material-induced reactant, the photochromic material-induced reactant, or the combination thereof, and can be used to achieve tattoo effects. A range outside the first region A1 preferably does not contain the thermochromic material-induced reactant, the photochromic material-induced reactant, or the combination thereof, but can contain the thermochromic material, the photochromic material, or the combination thereof. In the embodiment of FIG. 11, the base layer 420a of the first region A1 also contains the thermochromic material-induced reactant, the photochromic material-induced reactant, or the combination thereof; however, the present invention is not limited thereto. For example, the base layer 420a can be only composed of the biocompatible material without containing the thermochromic material, the photochromic material, the thermochromic material-induced reactant, the photochromic material-induced reactant, or the combination thereof.

As shown in an embodiment of FIG. 12, a material of a microneedle layer 400b can further include the tattoo pigment; that is, in addition to the thermochromic material, the photochromic material, the thermochromic material-induced reactant, the photochromic material-induced reactant, or the combination thereof, the tattoo pigment is additionally included. A method of constructing the microneedle layer 400b using the biocompatible material, the tattoo pigment, the thermochromic material, the photochromic material, the thermochromic material-induced reactant, the photochromic material-induced reactant, or the combination thereof can be as described above. In addition, the microneedle layer 400b can be manufactured by the same method as described above. It is known that the tattoo pigment can be distributed in a plurality of microneedles 410b and a base layer 420b, or only in the microneedles 410b.

The tattoo pigment can be uniformly distributed on all the microneedles 410b, or can be further distributed on the microneedles 410b of a first region A2. In some embodiments of the present invention, as shown in FIG. 12, a microneedle patch 40b can further have a pre-cut (dashed line in the figure) formed along the contour of the first region A2, for example, for a user to tear off the first region A2 of the microneedle patch 40b for use.

In some embodiments of the present invention, the microneedle patch, such as microneedle patches 40, 40a, and 40b, can further contain bioactive substances, such as transdermally deliverable molecules with pharmacological activity. Through the color change of the microneedle patches 40, 40a and 40b, such as color development, decolorization, or color conversion, not only can a user be reminded that the user is currently taking medication, but other people such as family members and medical staff can also be informed of the user's medication status. The color development, decolorization, or color conversion is not limited to being visible to the naked eyes but can be presented, for example, by a specific wavelength of light. Furthermore, the metabolism and/or absorption of certain drugs can be tracked from their color change status through a time-sensitive thermochromic material-induced reactant, a time-sensitive photochromic material-induced reactant, a time-sensitive tattoo pigment, or a combination thereof. For example, when the color becomes weaker, it can reflect the decrease in the effective dose of the drug, which can be used to remind the users to refill their medications.

In summary, the manufacturing method of the microneedle patch of the present invention is relatively simple, the cost is lower, and the process is more simplified than the current manufacturing methods of microneedle tattoo patches because of the use of the thermochromic material, the photochromic material, or the combination thereof, and further the combination with the mask along with the heat source, the cold source, or the light source. And, the mask can be easily produced with conventional techniques. Due to the simplified manufacturing process, it is beneficial to reducing the occurrence of errors, thereby improving the yield of microneedles; further, since the preparation process is simple, the cost is low, and the manufacturing process is simplified, it is beneficial to mass production of microneedle patches. On the other hand, because the material of the microneedle patch of the present invention includes the thermochromic material, the photochromic material, the thermochromic material-induced reactant, the photochromic material-induced reactant, or the combination thereof, the microneedle patch can be used for tattoos and can achieve decoration or marking effects.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A microneedle patch, comprising a microneedle layer, wherein:
the microneedle layer has a total area, the microneedle layer comprises a first region, and the first region has a first area less than or equal to the total area; and
a material of the microneedle layer comprises a biocompatible material, a thermochromic material, a photochromic material, a thermochromic material-induced reactant, a photochromic material-induced reactant, or a combination thereof.

2. The microneedle patch according to claim 1, wherein the microneedle layer further comprises a base layer and a plurality of microneedles, the base layer has a first side and a second side opposite to each other, the first region is located on the first side, and the microneedles are spaced apart from each other and arranged on the first side.

3. The microneedle patch according to claim 2, further comprising a carrier layer, wherein the microneedle layer is arranged on the carrier layer with the second side of the base layer.

4. The microneedle patch according to claim 1, wherein the thermochromic material, the photochromic material, the thermochromic material-induced reactant, the photochromic material-induced reactant, or the combination thereof is further distributed on a plurality of microneedles located in the first region.

5. The microneedle patch according to claim 1, wherein the thermochromic material comprises thermochromic liquid crystal, spironolactone, fluoroline, spiropyran, fulgide, triarylmethane, bis(methyldimethylamino)tetrachloronickelate (II), bis(ethyldiethylamino)tetrachlorocuprate (II), tetraorganodiarsenic, tetraorganoditin, tetraorganodibismuth, ammonium metavanadate, manganese violet, metal oxides, metal sulfides, iodides, or a combination thereof.

6. The microneedle patch according to claim 1, wherein the photochromic material comprises triarylmethane, spiropyran, spirooxazine, diarylethene, azobenzene, nitrone, fulgide, naphthopyrans, quinones, chlorides, halides, hydroxides, metal complexes, coordination compounds, dark color-changing minerals, or a combination thereof.

7. The microneedle patch according to claim 1, wherein the biocompatible material comprises hyaluronic acid, polyvinylpyrrolidone, polyvinyl alcohol, hydroxyethylcellulose, carboxymethylcellulose, hydroxypropylmethylcellulose, pullulan, collagen, xanthan gum, pectin, polyglutamic acid, short-chain polypeptide, sodium alginate, maltodextrin, cyclodextrin, propylene glycol alginate, gelatin, gum arabic, methylcellulose, polysaccharide, disaccharide, monosaccharide, polyol, or a combination thereof.

8. The microneedle patch according to claim 1, wherein an amount of the thermochromic material, the photochromic material, the thermochromic material-induced reactant, the photochromic material-induced reactant, or the combination thereof accounts for 0.1 to 40 wt % of the material of the microneedle layer.

9. The microneedle patch according to claim 8, wherein the amount of the thermochromic material, the photochromic material, the thermochromic material-induced reactant, the photochromic material-induced reactant, or the combination thereof accounts for 2 to 35 wt % of the material of the microneedle layer.

10. The microneedle patch according to claim 9, wherein the amount of the thermochromic material, the photochromic material, the thermochromic material-induced reactant, the photochromic material-induced reactant, or the combination thereof accounts for 10 to 25 wt % of the material of the microneedle layer.

11. The microneedle patch according to claim 1, wherein the material of the microneedle layer further comprises a tattoo pigment, and the amount of the thermochromic material, the photochromic material, the thermochromic material-induced reactant, the photochromic material-induced reactant, the tattoo pigment, or a combination thereof accounts for 0.1 to 40 wt % of the material of the microneedle layer.

12. The microneedle patch according to claim 11, wherein the tattoo pigment is further distributed on a plurality of microneedles located in the first region.

13. The microneedle patch according to claim 1, wherein the thermochromic material, the photochromic material, the thermochromic material-induced reactant, the photochromic material-induced reactant, or the combination thereof further are ingredients with tattoo effects.

14. The microneedle patch according to claim 1, wherein the first region has a pattern, and the first area of the first region is smaller than the total area.

15. A method for manufacturing a microneedle patch, the method comprising steps of:
providing a mold, wherein an internal space of the mold comprises a bottom surface, a plurality of tapered holes is formed on the bottom surface, and the tapered holes are spaced apart from each other and extend in a direction away from the bottom surface;
providing a biocompatible material, wherein the biocompatible material mixes with a thermochromic material, a photochromic material, or a combination thereof to form a first mixture;
dispensing the first mixture to the mold and forming a microneedle layer, wherein the microneedle layer comprises a plurality of microneedles spaced apart from each other;
arranging a mask above the microneedle layer, wherein the mask has a pattern region, and the pattern region is projected on the microneedle layer; and
providing a heat source, a cold source, a light source, or a combination thereof, and applying a temperature change, light, or a combination thereof to the microneedle layer across the mask so that the microneedle layer displays a pattern, wherein the pattern corresponds to the pattern region of the mask.

16. The method for manufacturing the microneedle patch according to claim 15, wherein an amount of the thermochromic material, the photochromic material, or the combination thereof accounts for 0.1 to 40 wt % of the first mixture.

17. The method for manufacturing the microneedle patch according to claim 15, wherein the step of providing the biocompatible material further comprises mixing a tattoo pigment into the first mixture and forming a second mixture, and the amount of the thermochromic material, the photochromic material, the tattoo pigment, or the combination thereof accounts for 0.1 to 40 wt % of the second mixture.

18. The method for manufacturing the microneedle patch according to claim 15, wherein the pattern region further is a hollow area on the mask.

19. The method for manufacturing the microneedle patch according to claim 15, wherein the step of applying the temperature change to the microneedle layer across the mask further comprises causing the thermochromic material to undergo a temperature change to produce a thermochromic material-induced reactant, and the step of applying the light to the microneedle layer across the mask further comprises causing the photochromic material to produce a photochromic material-induced reactant through illumination.
